(19) United States
(12) Patent Application Publication
(10) Pub. No.: US 2025/0025460 A1
(43) Pub. Date: Jan. 23, 2025

(54) USE OF A COMBINATION OF LEVOSIMENDAN AND AN SGLT-2 INHIBITOR TO TREAT HEART FAILURE

(71) Applicant: Tenax Therapeutics, Inc., Chapel Hill, NC (US)

(72) Inventors: Stuart Rich, Skokie, IL (US); Douglas Randall, Wake Forest, NC (US)

(73) Assignee: Tenax Therapeutics, Inc., Chapel Hill, NC (US)

(21) Appl. No.: 18/714,318

(22) PCT Filed: Nov. 30, 2022
(86) PCT No.: PCT/US2022/080708

§ 371 Date: May 29, 2024

Related U.S. Application Data
(60) Provisional application No. 63/285,298, filed on Dec. 2, 2021.

(51) Int. Cl. A61K 31/50; A61K 31/357; A61K 31/7048; A61P 9/04

(52) U.S. Cl. CPC A61K 31/50; A61K 31/357; A61K 31/7048; A61P 9/04

(57) ABSTRACT

This invention relates to a method of treating heart failure in a human subject comprising administering to the human subject an effective amount of a combination therapy comprising an amount of levosimendan, its metabolites OR-1896 or OR-1855, or a combination thereof; and an amount of a sodium-glucose cotransporter-2 (SGLT-2) inhibitor.

This application claims the benefit of U.S. Provisional Application No. 63/285,298, filed Dec. 2, 2021, the contents of which are hereby incorporated by reference.

Throughout this application, various publications are referenced, including referenced in parenthesis. The disclosures of all publications mentioned in this application in their entireties are hereby incorporated by reference into this application in order to provide additional description of the art to which this invention pertains and of the features in the art which can be employed with this invention.

### BACKGROUND OF THE INVENTION

### Levosimendan

Levosimendan is a calcium sensitizer and potassium channel activator drug approved in over 60 countries for use in subjects with acutely decompensated heart failure (ADHF).

Levosimendan enhances the calcium sensitivity of contractile proteins by binding to cardiac troponin C in a calcium-dependent manner. Levosimendan increases the contraction force but does not impair ventricular relaxation. In addition, levosimendan opens ATP-sensitive potassium channels in vascular smooth muscle, thus inducing vasodilatation of systemic and coronary arterial resistance vessels and systemic venous capacitance vessels. Levosimendan is also a selective phosphodiesterase III inhibitor in vitro. (Simdax. Finland: Orion Corporation; 2010)

Levosimendan's activity is mediated through unique mechanisms of action, including increased cardiac contractility by calcium sensitization of troponin C, vasodilation through opening of potassium channels, and cardioprotective effects via potassium channel opening in mitochondria. (Haikala et al. 1995, Haikala et al. 1995, Pollesello et al. 1994, Sorsa et al. 2004, Yokoshiki et al. 1997, Pataricza et al. 2000, Kaheinen et al. 2001, Erdei et al. 2006, Maytin et al. 2005, Pollesello et al. 2007, du Toit et al. 2008, Louhelainen et al. 2010)

Levosimendan has been shown to be a potent and selective phosphodiesterase-3 (PDE3) inhibitor in vitro. The drug is PDE3 selective with a PDE3/PDE4 inhibition ratio of 10,000. However, both isozymes must be inhibited in cardiomyocytes to exert an effect on the cAMP concentration and inotropic effects. The classical PDE inhibitors (i.e., milrinone, enoximone, and amrinone) inhibit both PDE3 vs. PDE4 is as low as 17-fold), which accounts fully for their inotropic effect. (Yokoshiki et al. 1997, Szilagyi et al. 2004)

Levosimendan improves endothelial function and enhances diastolic coronary flow by opening the adenosine triphosphate-sensitive potassium channels and increasing nitric oxide production. Levosimendan acts through direct binding to troponin-C at high systolic intracellular calcium concentration as well as detachment from it at low diastolic concentration are facilitated. Levosimendan displayed positive lusitropic effects relative to milrinone and nitroglycerin. The lusitropic effect of levosimendan is independent of the degree of the inotropic effect. (Michaels et al. 2005, Grossini et al. 2005, Hasenfuss et al. 1998, De Luca et al. 2006)

### Metabolites OR-1896 and OR-1855

Levosimendan has an active metabolite that extends its effects well beyond the infusion period. Following intravenous or oral dosing, levosimendan is reduced by intestinal bacteria to form OR-1855 (limited activity) that is acetylated to form OR-1896, an active metabolite. While the patient half-life is approximately one hour and cleared a few hours after the end of intravenous infusion, OR-1896 has a prolonged half-life of 70-80 hours in heart failure subjects with roughly equal exposures of OR-1855 and OR-1896 maintained through deacetylation/acetylation pathways. The OR-1896 metabolite has been shown to retain similar hemodynamic and pharmacologic properties of levosimendan and maintain roughly equivalents to levosimendan in preclinical models. This activity occurs despite considerably lower plasma concentrations relative to levosimendan, an apparent result of a large percentage of unbound OR-1896 in circulation. Thus, in extended repeated dosing, levosimendan is essentially an active prodrug to an active metabolite moiety, OR-1896. (Louhelainen et al. 2010, Erdei et al. 2006, Szilagyi et al. 2004, Banfor et al. 2008, Louhelainen et al. 2009, Segreti et al. 2008)

OR-1896 is equipotent to levosimendan in its inotropic effects in whole cardiomyocytes and isolated contractile apparatus preparations. However, OR-1896 is profoundly less potent in the inhibition of both PDE3 and PDE4 isozymes. This supports the hypothesis that the main component of the inotropic effect for both levosimendan and OR-1896 is a result of their binding to troponin C and not through PDE inhibition. (Szilagyi et al. 2004)

Clinical observations demonstrate that short-term levosimendan administration is followed by long-term hemodynamic changes that parallel the levels of OR-1896. Patients have been observed with detectable concentrations of both metabolites, OR-1896 and OR-1855, in follow-ups two weeks after treatment. Despite OR-1855's observed inactivity, OR-1896 greatly extends the parent levosimendan's activity and provides the primary active moiety in subjects receiving intermittent intravenous levosimendan therapy. (Banfor et al. 2007, Kivikko et al. 2003, Kivikko et al. 2002)

Based on knowledge of OR-1896 and OR-1855, administration of the metabolites could be used analogously to levosimendan, with adjustments made for the metabolites' own parameters. Both metabolites could be delivered through various routes of administration, including but not limited to, oral, intravenous, and subcutaneous administration. The dose chosen depends on the specific route of administration. In all cases, the target dosing would be intended to achieve a steady-state concentration of OR-1896 of 0.5 to 10.0 ng/ml. The relationship between levosimendan and OR-1896 and OR-1855, along with the interaction between the metabolites, is discussed in “Pharmacodynamics and Safety of a New Calcium Sensitizer. Levosimendan, and Its Metabolites during an Extended Infusion in Patients with Severe Heart Failure” (Kivikko et al. 2002), the entire contents of which are incorporated by reference.

### Sodium-Glucose Cotransporter 2 (SGLT-2) Inhibitors

SGLT-2 inhibitors, also referred to as gliflozins, are typically used to lower high blood glucose levels in people with type 2 diabetes. SGLT-2 inhibitors inhibit SGLT-2 proteins located in the renal tubules of the kidneys which are responsible for reabsorbing glucose back into the blood. As a result, more glucose is excreted in the urine. SGLT-2 inhibitors have been shown to be effective at lowering hemoglobin A1c levels, improving weight loss, and lowering blood pressure.

SGLT-2 inhibitors have also been proven to be safe, effective, and FDA approved for the treatment of heart failure, although the precise mechanism employed to achieve positive results for heart failure is not known. See. for example. U.S. Patent Application Publication Nos. 2020/0078382 A1 and US 2020/0360412 A1, the entire contents of each of which are incorporated by reference. Additionally, the EMPERIAL-Preserved and EMPERIAL-Reduced clinical trials studied the effect of empagliflozin on heart failure, including the drug's effect on exercise capacity. It is noteworthy that neither of these trials showed an improvement in exercise capacity as measured by change in six-minute walk distance (Abraham et al.). Similarly, another clinical trial. EMBRACE HF, showed that pulmonary hypertension with heart failure with preserved ejection fraction (PH-HFpEF) patients receiving empagliflozin alone did not demonstrate a meaningful difference in 6-minute walk distance or improved quality of life (Nassif et al.).

### Types of Heart Failure

Heart failure encompasses several different indications, including heart failure with preserved ejection fraction (HFpEF) and heart failure with reduced ejection fraction (HFrEF), which are distinct clinical entities. While HFpEF and HFrEF types of heart failure account for approximately 50% of all heart failure patients, many differences exist between these two forms of heart failure. A recent review by Shaw et al. described some of the distinct features of HFpEF and HFrEF. See also PCT International Application Publication No. WO 2021/126884, the entire contents of which are incorporated by reference.

### Combination Therapy

The administration of two drugs to treat a given condition, such as the combination of levosimendan and an SGLT-2 inhibitor to treat heart failure, raises a number of potential problems. In vivo interactions between two drugs are complex. The effects of any single drug are related to its absorption, distribution, and elimination. When two drugs are introduced into the body, each drug can affect the absorption, distribution, and elimination of the other and hence, alter the effects of the other. For instance, one drug may inhibit, activate or induce the production of enzymes involved in a metabolic route of elimination of the other drug (Guidance for Industry, 2006). In one example, combined administration of GA and interferon (IFN) has been experimentally shown to abrogate the clinical effectiveness of either therapy. (Brod 2000) In another experiment, it was reported that the addition of prednisone in combination therapy with IFN-β antagonized its up-regulator effect. Thus, when two drugs are administered to treat the same condition, it is unpredictable whether each will complement, have no effect on, or interfere with, the therapeutic activity of the other in a human subject.

Not only may the interaction between two drugs affect the intended therapeutic activity of each drug, but the interaction may increase the levels of toxic metabolites (Guidance for Industry, 2006). The interaction may also heighten or lessen the side effects of each drug. Hence, upon administration of two drugs to treat a disease, it is unpredictable what change will occur in the negative side profile of each drug. In one example, the combination of natalizumab and interferon B-la was observed to increase the risk of unanticipated side effects. (Vollmer, 2008; Rudick 2006; Kleinschmidt-DeMasters, 2005; Langer-Gould 2005)

Additionally, it is difficult to accurately predict when the effects of the interaction between the two drugs will become manifest. For example, metabolic interactions between drugs may become apparent upon the initial administration of the second drug, after the two have reached a steady-state concentration or upon discontinuation of one of the drugs. (Guidance for Industry, 2006)

The combination of levosimendan and an SGLT-2 inhibitor has never previously been studied for the treatment of any type of heart failure, including HFpEF or HFrEF populations with or without accompanying pulmonary hypertension. The EMPERIAL clinical trials studying the effect of empagliflozin on heart failure specifically excluded patients receiving inotropic drugs, such as levosimendan. Indeed, such a combination may have been considered potentially harmful to such patients because the separate use of the SGLT-2 inhibitor empagliflozin alone and levosimendan alone have been associated with hypotension. Accordingly, the state of the art at the time of filing is that the effects of combination therapy of two drugs, in particular levosimendan and an SGLT-2 inhibitor, cannot be predicted until the results of a combination study are available.

### SUMMARY OF THE INVENTION

This invention provides a method of treating heart failure in a human subject comprising administering to the human subject an effective amount of a combination therapy comprising an amount of levosimendan, its metabolites OR-1896 or OR-1855, or a combination thereof; and an amount of a sodium-glucose cotransporter-2 (SGLT-2) inhibitor.

This invention also provides pharmaceutical composition comprising an amount of a SGLT-2 inhibitor and an amount of levosimendan, its metabolites OR-1896 or OR-1855, or a combination thereof, for use in treating a subject afflicted with heart failure, wherein the SGLT-2 inhibitor and the levosimendan, its metabolites OR-1896 or OR-1855, or a combination thereof, are administered simultaneously, contemporaneously or concomitantly.

Accordingly, the invention provides a combination of levosimendan and a SGLT-2 inhibitor, and medicaments comprising the combination, for use in treating heart failure. Other objects, features and advantages of the present invention will become clear from the following description and drawings.

### BRIEF DESCRIPTION OF THE DRAWINGS

FIG. 1: Pulmonary Capillary Wedge Pressure (PCWP) of Patient 019-022 of HELP Study. Patient 019-022 showed a decrease in pulmonary capillary wedge pressure (PCWP) after levosimendan administration. These results are based on the HELP Study single 24-hour open label lead-in infusion data.

FIG. 2: 6-Minute Walk Distance of Patient 019-022 of HELP Study. Patient 019-022 showed a significant improvement in their 6-minute walk distance after receiving a combination therapy of levosimendan and empagliflozin.

FIG. 3: 6-Minute Walk Distance Change during single agent therapy (Levosimendan alone) vs. combination therapy of Empagliflozin and Levosimendan. Data from the HELP Study and Patient 019-002 are shown to compare the effects of treatment with levosimendan alone and the combination of empagliflozin and levosimendan on exercise capacity as measured by 6-minute walk distance changes.

### DETAILED DESCRIPTION OF THE INVENTION

According to some embodiments, the invention provides a method of treating heart failure in a human subject comprising administering to the human subject an effective amount of a combination therapy comprising a) an amount of levosimendan, its metabolites OR-1896 or OR-1855, or a combination thereof; and b) an amount of a sodium-glucose cotransporter-2 (SGLT-2) inhibitor.

In some embodiments, the method comprises periodically administering to the subject an amount of the levosimendan, its metabolites OR-1896 or OR-1855, or a combination thereof, and an amount of the SGLT-2 inhibitor, wherein the amounts when taken together are effective to treat the subject.

In some embodiments, treating the subject with the combination therapy is more effective to treat the subject than when either the amount of levosimendan or the amount of the SGLT-2 inhibitor is administered alone.

In some embodiments, the amounts of levosimendan and the SGLT-2 inhibitor when taken together are effective to achieve a greater than additive therapeutic result in treating the subject.

In some embodiments, the subject was receiving a therapy including levosimendan, its metabolites OR-1896 or OR-1855, or a combination thereof, prior to initiating a SGLT-2 inhibitor therapy.

In some embodiments, the subject was receiving a SGLT-2 inhibitor therapy prior to initiating a therapy including levosimendan, its metabolites OR-1896 or OR-1855, or a combination thereof.

In some embodiments, the amount of SGLT-2 inhibitor is administered first, followed by administration of the amount of levosimendan, its metabolites OR-1896 or OR-1855, or a combination thereof.

In some embodiments, the amount of levosimendan, its metabolites OR-1896 or OR-1855, or a combination thereof, is administered first, followed by administration of a SGLT-2 inhibitor.

In some embodiments, the levosimendan, its metabolites OR-1896 or OR-1855, or a combination thereof, and the SGLT-2 inhibitor are administered sequentially.

In some embodiments, the levosimendan, its metabolites OR-1896 or OR-1855, or a combination thereof, and the SLGT-2 inhibitor are administered simultaneously.

In some embodiments, the levosimendan, its metabolites OR-1896 or OR-1855, or a combination thereof, and the SLGT-2 inhibitor are administered periodically, chronically, weekly, or intermittently.

In some embodiments, the SGLT-2 inhibitor is administered orally.

In some embodiments, the SGLT-2 inhibitor is selected from the group consisting of empagliflozin, canagliflozin, ertugliflozin, or dapagliflozin.

In some embodiments, the subject is administered between 10-25 mg empagliflozin per day.

In some embodiments, the subject is administered between 5-10 mg dapagliflozin per day.

In some embodiments, the subject is administered between 100-300 mg canagliflozin per day.

In some embodiments, the subject is administered between 5-15 mg ertugliflozin per day.

In some embodiments, the levosimendan, its metabolites OR-1896 or OR-1855, or a combination thereof, is administered orally, intravenously, or subcutaneously.

In some embodiments, the subject is administered between 0.1-10 mg of levosimendan, its metabolites OR-1896 or OR-1855, or a combination thereof, per day, preferably between 1-4 mg of levosimendan, its metabolites OR-1896 or OR-1855, or a combination thereof per day.

In some embodiments, the combination therapy is administered as a fixed dose combination.

In some embodiments, the heart failure is heart failure with preserved ejection fraction (HFpEF) or heart failure with reduced ejection fraction (HFrEF).

In some embodiments, the heart failure is accompanied by pulmonary hypertension.

In some embodiments, the heart failure accompanied by pulmonary hypertension is pulmonary hypertension with heart failure with preserved ejection fraction (PH-HFpEF) or pulmonary hypertension with heart failure with reduced ejection fraction (PH-HFrEF).

In some embodiments, the treating with the combination therapy comprises providing
a) an improvement in the human subject's quality of life;
b) an improvement in the human subject's exercise capacity;
c) an improvement in a physician's assessment of the human subject's functional class; and/or
d) a reduction in the incidence of hospitalization for heart failure.

In some embodiments, the treating with the combination therapy comprises providing an improvement in the human subject's exercise capacity.

In some embodiments, the improvement in the subject's exercise capacity is an increase of at least 10, 20, 30, 40, 50, 60, 70, 80, or 100 meters in a 6-minute walk distance compared to a baseline 6-minute walk distance before the combination therapy treatment.

In some embodiments, the improvement in the subject's exercise capacity is an increase of at least 10%, 20%, 30%, 40%, or 50% relative to a baseline 6-minute walk distance before the combination therapy treatment.

In some embodiments, the improvement in the subject's exercise capacity is within one, two, three, four, five, six, seven, eight, nine, ten, twenty, thirty, forty, or fifty weeks of the administration of the combination therapy.

In some embodiments, the treating comprises providing an improvement in the human subject's hemodynamic measurements at rest and exercise.

According to some embodiments, the invention provides a pharmaceutical composition comprising levosimendan, its metabolites OR-1896 or OR-1855, or a combination thereof, a SGLT-2 inhibitor, and a pharmaceutically acceptable carrier.

In some embodiments, the SGLT-2 inhibitor is empagliflozin.

According to some embodiments, the invention provides use of a SGLT-2 inhibitor in combination or as an add-on with a therapy that includes levosimendan, its metabolites OR-1896 or OR-1855, or a combination thereof, to treat a subject afflicted with heart failure, wherein the SGLT-2 inhibitor and the levosimendan, its metabolites OR-1896 or OR-1855, or a combination thereof are administered simultaneously, contemporaneously or concomitantly.

In some embodiments, the SGLT-2 inhibitor is selected from the group consisting of empagliflozin, canagliflozin, ertugliflozin, or dapagliflozin.

In some embodiments, the heart failure is heart failure with preserved ejection fraction (HFpEF), heart failure with reduced ejection fraction (HFrEF), pulmonary hypertension with heart failure with preserved ejection fraction (PH-HFpEF) or pulmonary hypertension with heart failure with reduced ejection fraction (PH-HFrEF).

According to some embodiments, the invention provides use of a SGLT-2 inhibitor in the manufacturing of a medicament for use in combination with or as an add-on to a therapy that includes levosimendan, its metabolites OR-1896 or OR-1855, or a combination thereof, to treat a subject afflicted with heart failure, wherein the SGLT-2 inhibitor and the levosimendan, its metabolites OR-1896 or OR-1855, or a combination thereof are administered simultaneously, contemporaneously or concomitantly.

In some embodiments, the heart failure is heart failure with preserved ejection fraction (HFpEF), heart failure with reduced ejection fraction (HFrEF), pulmonary hypertension with heart failure with preserved ejection fraction (PH-HFpEF) or pulmonary hypertension with heart failure with reduced ejection fraction (PH-HFrEF).

In some embodiments, the SGLT-2 inhibitor is selected from the group consisting of empagliflozin, canagliflozin, ertugliflozin, or dapagliflozin.

According to some embodiments, the invention provides a pharmaceutical composition comprising an amount of a SGLT-2 inhibitor and an amount of levosimendan, its metabolites OR-1896 or OR-1855, or a combination thereof, for use in treating a subject afflicted with heart failure, wherein the SGLT-2 inhibitor and the levosimendan, its metabolites OR-1896 or OR-1855, or a combination thereof, are administered simultaneously, contemporaneously or concomitantly.

In some embodiments, the heart failure is heart failure with preserved ejection fraction (HFpEF), heart failure with reduced ejection fraction (HFrEF), pulmonary hypertension with heart failure with preserved ejection fraction (PH-HFpEF) or pulmonary hypertension with heart failure with reduced ejection fraction (PH-HFrEF).

In some embodiments, the SGLT-2 inhibitor is selected from the group consisting of empagliflozin, canagliflozin, ertugliflozin, or dapagliflozin.

According to some embodiments, the invention provides a package comprising:
a first pharmaceutical composition comprising an amount of levosimendan and a pharmaceutically acceptable carrier;
a second pharmaceutical composition comprising an amount of an SGLT-2 inhibitor and a pharmaceutically acceptable carrier; and
instructions for use of the first and second pharmaceutical compositions together to treat a subject afflicted with heart failure.

In some embodiments, the SGLT-2 inhibitor is empagliflozin and the heart failure is heart failure with preserved ejection fraction (HFpEF), heart failure with reduced ejection fraction (HFrEF), pulmonary hypertension with heart failure with preserved ejection fraction (PH-HFpEF) or pulmonary hypertension with heart failure with reduced ejection fraction (PH-HFrEF).

According to some embodiments, the invention provides a pharmaceutical composition in unit dosage form, useful in treating a subject afflicted with heart failure, which comprises an amount of levosimendan, and an amount of an SGLT-2 inhibitor, wherein the respective amounts of said levosimendan and said SGLT-2 inhibitor in said composition are effective, upon concomitant administration to said subject of one or more said unit dosage forms of said composition, to achieve a greater than additive therapeutic result in treating the subject.

In some embodiments, the SGLT-2 inhibitor is selected from the group consisting of empagliflozin, canagliflozin, ertugliflozin, or dapagliflozin, and the heart failure is heart failure with preserved ejection fraction (HFpEF), heart failure with reduced ejection fraction (HFrEF), pulmonary hypertension with heart failure with preserved ejection fraction (PH-HFpEF) or pulmonary hypertension with heart failure with reduced ejection fraction (PH-HFrEF).

According to some embodiments, the invention provides a therapeutic package for dispensing to, or for use in dispensing to, a subject afflicted with heart failure, which comprises:
one or more unit doses, each such unit dose consisting essentially of:
an amount of levosimendan; and
an amount of an SGLT-2 inhibitor,
wherein the respective amounts of said levosimendan and said SGLT-2 inhibitor in said unit dose are effective, upon concomitant administration to said subject, to achieve a greater than additive therapeutic result in treating the subject, and
a finished pharmaceutical container therefor, said container containing said unit dose or unit doses, said container further containing or comprising labeling directing the use of said package in the treatment of said subject.

In some embodiments, the SGLT-2 inhibitor is selected from the group consisting of empagliflozin, canagliflozin, ertugliflozin, or dapagliflozin, and the heart failure is heart failure with preserved ejection fraction (HFpEF), heart failure with reduced ejection fraction (HFrEF), pulmonary hypertension with heart failure with preserved ejection fraction (PH-HFpEF) or pulmonary hypertension with heart failure with reduced ejection fraction (PH-HFrEF).

Throughout this application, where a parameter range is provided, all integers within that range, and tenths and hundredths thereof as appropriate, shall be considered to also be provided and disclosed in this application as being contemplated by the invention. For example, “0.2-5 mg/kg/day” is to be considered as a disclosure of 0.2 mg/kg/day, 0.3 mg/kg/day, 0.4 mg/kg/day, 0.5 mg/kg/day, 0.6 mg/kg/day etc. up to 5.0 mg/kg/day.

According to some embodiments, the combination to be administered (e.g. levosimendan and a SGLT-2 inhibitor) is in the form of a composition comprising a therapeutically effective amount of at least one of the combination. As used herein, the term “effective amount” means an amount of each agent of the combination that is capable of reducing and/or attenuating a disorder or symptom as described herein. The specific dose of a compound administered according to this invention will, of course, be determined by the particular circumstances surrounding the case including, for example, the compound administered, the route of administration, the physiological state of the subject, and the severity of the condition being treated.

Any suitable route may be used to administer the medicament of the invention to a subject.

According to some embodiments, suitable administration routes may be systemic routes. According to some embodiments, administering is administering systemically. According to some embodiments, the composition is formulated for systemic administration. According to some embodiments, the composition is formulated for intravenous or subcutaneous administration.

According to another embodiment, administration systemically is through an enteral route. According to another embodiment, administration through an enteral route is oral administration. According to some embodiments, the composition is formulated for oral administration.

### Definitions/Abbreviations

As used herein, the term “levosimendan” means levosimendan base or a pharmaceutically acceptable salt thereof. The active compounds for use according to the invention may be provided in any form suitable for the intended administration. Suitable forms include pharmaceutically (i.e. physiologically) acceptable salts, and pre- or prodrug forms of the compound of the invention.

Examples of pharmaceutically acceptable addition salts include, without limitation, the non-toxic inorganic and organic acid addition salts such as the hydrochloride, the hydrobromide, the L-tartrate, the nitrate, the perchlorate, the phosphate, the sulphate, the formate, the acetate, the aconate, the ascorbate, the benzenesulphonate, the benzoate, the cinnamate, the citrate, the embonate, the enantate, the fumarate, the glutamate, the glycolate, the lactate, the maleate, the malonate, the mandelate, the methanesulphonate, the naphthalene-2-sulphonate, the phthalate, the salicylate, the sorbate, the stearate, the succinate, the tartrate, the toluene-p-sulphonate, and the like. Such salts may be formed by procedures well known and described in the art.

HFpEF is the abbreviation for heart failure with preserved ejection fraction. HFpEF is when a patient is afflicted with heart failure while their ejection fraction remains ≥40%. (Kelly et al. 2015)

HFrEF is the abbreviation for heart failure with reduced ejection fraction.

PH is the abbreviation for Pulmonary Hypertension. PH encompasses a heterogeneous group of disorders with the common feature of elevated pulmonary vascular resistance. (Oldroyd et al. 2019)

PH-HFpEF is the abbreviation for Pulmonary Hypertension with heart failure with preserved ejection fraction. PH-HFpEF is defined by a high pulmonary artery pressure, high left ventricular end-diastolic pressure and a normal ejection fraction. (Lai et al. 2019)

As used herein, “combination” means an assemblage of reagents for use in therapy either by simultaneous, contemporaneous, or fixed-dose combination delivery. Simultaneous delivery refers to delivery of an admixture (whether a true mixture, a suspension, an emulsion or other physical combination) of the drugs. In this case, the combination may be the admixture or separate containers of the levosimendan and a SGLT-2 inhibitor that are combined just prior to delivery. Contemporaneous delivery refers to the separate delivery of the levosimendan and SGLT-2 inhibitor at the same time, or at times sufficiently close together that an additive or preferably synergistic activity relative to the activity of either the levosimendan or the SGLT-2 inhibitor alone is observed. Fixed-dose combination delivery refers to the delivery of two or more drugs contained in a single dosage form for oral administration, such as a capsule or tablet.

Each agent of the combination can be administered in an effective dose and regiment suitable to treat heart failure as part of the combination. For instance, non-limiting examples of SGLT-2 inhibitor dosing include: a starting dose of 100 mg canagliflozin daily, with a maximum dose of 300 mg daily; a starting dose of 5 mg dapagliflozin daily, with a maximum dose of 10 mg daily; or a starting dose of 10 mg empagliflozin daily, with a maximum dose of 25 mg daily.

A combination therapy refers to several drugs being substantially effective in the body at a same time. Several drugs can be administered substantially at the same time, or can be administered at different times but have effect on the body at the same time. For example, this includes administering levosimendan before or subsequently to an SGLT-2 inhibitor, while functioning of levosimendan in the body is substantially extant.

PCWP is the abbreviation for pulmonary capillary wedge pressure. PCWP is the pressure measured by wedging a pulmonary catheter with an inflated balloon into a small pulmonary arterial branch. PCWP estimates left atrial pressure. (Peacock et al. 2004)

RAP is the abbreviation for right atrial pressure. RAP is the blood pressure in the right atrium of the heart. RAP reflects the amount of blood returning to the heart and the ability of the heart to pump the blood into the arterial system.

mPAP is the abbreviation for mean pulmonary artery pressure. mPAP is generated by the right ventricle ejecting blood into the pulmonary circulation, which acts as a resistance to the output from the right ventricle.

PVR is the abbreviation for pulmonary vascular resistance. PVR refers to the resistance in the arteries that supply blood to the lungs. (Schnur 2017)

CO is the abbreviation for cardiac output. CO is the volume of blood being pumped by the heart per unit time. (Vincent 2008)

CI is the abbreviation for cardiac index. CI is a hemodynamic parameter that relates the cardiac output from the left ventricle in one minute to the body surface area. This measurement relates heart performance to the size of the individual. (Shea 2019)

HR is the abbreviation for heart rate. HR is the speed of the heartbeat measured by the number of contractions of the heart per minute. (Heart.org 2015)

PR is the abbreviation for pulse rate. PR is the measurement of the heart rate. (Heart.org 2015)

BP is the abbreviation for blood pressure. BP is the pressure of circulating blood within the major arterial system of the body. (Brezinski 1990)

6 MWT is the abbreviation for six-minute walk test. 6 MWT is a performance-based test used to measure functional exercise capacity. The 6 MWT measures the distance an individual is able to walk over a total of 6 minutes at a constant and normal pace. (Vandoni et al. 2018)

Likert scale is a psychometric scale commonly involved in research that employs questionnaires. In the below-mentioned clinical trial, a six-question, five-point Likert Scale is provided to patients to assess their quality of life. (HELP clinical trial protocol)

ECG is the abbreviation for echocardiogram. An ECG is a record of a person's heartbeat produced by echocardiography. An ECG is a test that uses high frequency sound waves (ultrasound) to make pictures of your heart. (heart.org 2015)

Dobutamine stress test is a form of ECG where stress is induced on the heart by administering dobutamine into a vein to assess the heart's function and structures. This test mimics the effects of exercise on the heart. (Hawthorne et al. 2012)

New York Heart Association Functional Classification provides a simple way of classifying the extent of heart failure. A patient in Class I has no limitation of physical activity. A patient in Class II has slight limitation of physical activity. A patient in Class III has marked limitation of physical activity. A patient in Class IV is unable to carry on any physical activity without discomfort. In addition to these class numbers based of patient symptoms, every patient is assigned a class letter based on an objective assessment. A patient in Class A has no objective evidence of cardiovascular disease. A patient in Class B has objective evidence of minimal cardiovascular disease. A patient in Class C has objective evidence of moderately severe cardiovascular disease. A patient in Class D has objective evidence of severe cardiovascular disease. (Yancy et al. 2013)

Self-administration is administration of the formulation administered by the human subject afflicted with the disease. (HELP clinical trial protocol)

Outpatient setting is a setting where the patients do not require admittance for overnight care. (World Health Organization 2009)

Trained professional indicates a doctor, nurse, home healthcare nurse, or other person with training and/or experience and/or a license in the medical profession.

TEAEs is the abbreviation for Treatment Emergent Adverse Events. The TEAEs of special interest are hypotension, atrial fibrillation, other significant arrhythmia, resuscitated death stroke. Other TEAEs include, but are not limited to, headache, increased heart rate, fatigue, cardiac failure acute, dyspnea, vascular access site pain, muscle spasm, and hypokalaemia.

SAEs is the abbreviation for Serious Adverse Events. SAEs include, but are not limited to, infections and infestations, device related infection; infections and infestations, bacteremia; cardiac disorders, cardiac failure acute; and cardiac disorders, cardiac failure acute.

As used herein, the term “acute administration” means administration of a drug in a brief period of time, for example, delivery of a single dose of a drug, delivery of doses of a drug in rapid succession, or delivery of a drug on short time scale, preferably less than 48 hours. Acute administration of a drug is generally intended for the drug to have a beneficial effect on a condition in the short-term. For example, acute administration of levosimendan may be performed such that the amount of levosimendan administered is intended for the levosimendan drug to directly improve a condition prior to any significant activity of a levosimendan metabolite. Acute administration is generally performed by a trained professional, for example, by intravenous administration in a clinical setting.

As used herein, the term “chronic administration” means an extended and repeated administration of a drug. For example, delivery of multiple or repeated doses of a drug over the course of a long-time scale, preferably at least a week. Chronic administration of a drug is generally intended for the drug or its metabolites to have a continued beneficial effect on a condition or to prevent or slow deterioration of a disease-state over time. Chronic administration is often delivered to a subject by the subject themselves (i.e. self-administration), for example, by oral or subcutaneous administration.

### Subcutaneous Administration of Levosimendan

The combination of levosimendan and SGLT-2 includes subcutaneous administration of levosimendan in a subcutaneous formulation to achieve the effects disclosed herein. Sec, for example, PCT International Application No. WO/2020/041180 A1, the entire contents of which is incorporated by reference.

Notwithstanding the potential extravasation concerns with subcutaneous administration, the subcutaneous route of levosimendan administration may be better tolerated than intravenous delivery. Subcutaneous administration may reduce and delay absorption of levosimendan, resulting in lower peak plasma concentrations of levosimendan as compared to intravenous administration. This may avoid the occurrence of typical side effects of levosimendan administration, particularly hypotension caused by the maximum concentration of levosimendan (Cmax) because higher plasma concentrations and a higher Cmax of levosimendan typically leads to more frequent side effects, such as hypotension.

Subcutaneous administration of levosimendan offers a clear advantage in terms of eliminating the potential for central line infections that are common with IV chronic administration via PICC and port-a-cath devices that are often necessary for convenient repeated IV access.

Despite exhibiting a delay in levosimendan absorption along with lower plasma concentrations, subcutaneous administration of levosimendan unexpectedly results in a similar plasma concentration profile of the levosimendan metabolite OR-1896. This results in a better safety profile, due to the comparable levels of OR-1896 in the blood without the high peak plasma concentration of levosimendan.

There are numerous practical advantages with subcutaneous administration such as: being easily titrated, facilitating patient control, reliable records of dosing, reducing nursing burden, and reducing the risk of drug diversion.

One delivery device suitable for subcutaneous administration is an ambulatory infusion pump, such as a CADD pump, which stands for continuous ambulatory delivery device pump. Additionally, subcutaneous administration could be delivered through a simple prefilled syringe, syringe pump, injection pen, autoinjector, micropump, or patch device. (Bittner et al. 2018)

Although in the present instance the subcutaneous formulation would be substantially similar to the intravenous formulation, certain additives could be introduced to make the treatment more tolerable for the patient. Buffers, such as water, sodium bicarbonate, or other similar buffering agents that are known to increase pH, could be added to increase the pH of the subcutaneous formulation.

Like the administration of the intravenous formulation, the subcutaneous administration of levosimendan can be administered intermittently as well as chronically. The intermittent administration can take place weekly for a 24-hour period. In addition to being administered by a trained professional, subcutaneous administration makes it much simpler for the patient to self-administer the levosimendan formulation.

Subcutaneous administration of levosimendan can support an expanded range of dilutions and corresponding concentration while still being viable.

| | | | | | | Small Volume |
|---|---|---|---|---|---|---|
| Example | | | | | 24 Hr | (0.1-2.0 ml) via |
| Simdax | | Vol | Total | Diluted | continuous | Daily injection, |
| Vial | Vial | Diluent | Vol | Concentration | Infusion | Pump, or Patch |
| (mg) | Vol/ml | ml | ml | mg/ml | Rate ml/hr | Delivery |
| 12.5 | 5 | 0 | 5 | 2.5000 | 0.21 | 0.1-2.0 |
| 12.5 | 5 | 5 | 10 | 1.2500 | 0.42 | 0.5-2.0 |
| 12.5 | 5 | 25 | 30 | 0.4167 | 1.25 | NA |
| 12.5 | 5 | 75 | 80 | 0.1563 | 3.33 | NA |
| 12.5 | 5 | 150 | 155 | 0.0806 | 6.46 | NA |
| 12.5 | 5 | 250 | 255 | 0.0490 | 10.63 | NA |
| 12.5 | 5 | 500 | 505 | 0.0248 | 21.04 | NA |
| 12.5 | 5 | 1000 | 1005 | 0.0124 | 41.88 | NA |
| 12.5 | 5 | 1500 | 1505 | 0.0083 | 62.71 | NA |

Overall, data supports that subcutaneous administration is simpler than intravenous infusions, can reduce drug delivery-related healthcare costs and resources, and is largely preferred by both patients and health care providers (Bittner et al. 2018).

Several methods of subcutaneous administrations are known in the art, and any such method may be used for subcutaneous administration of levosimendan. Examples of subcutaneous administration methods include, but are not limited to, manual needle injection and various subcutaneous drug delivery devices such as subcutaneous administration systems that deliver a drug via a pump apparatus such as performed with certain insulin pump systems, e.g. the Omnipod@ system.

In an embodiment the subcutaneous formulation comprises water in an amount effective to reduce pain caused by the administration.

In an embodiment the subcutaneous formulation comprises buffers to raise the pH higher than 3.0.

In an embodiment the subcutaneous administration reduces side effects relative to intravenous administration.

In an embodiment the subcutaneous administration reduces peak plasma concentrations of levosimendan relative to intravenous administration by at least 1% to 25%.

Levosimendan formulations that have been described for subcutaneous administration are described in, for example, PCT International Publication No. WO 2020/041180 A1 (Application No. PCT/US2019/047032), the entire contents of which are incorporated by reference.

In some embodiments, a pharmaceutical composition of levosimendan for treatment in subjects in need thereof by subcutaneous administration is in a formulation comprising: (a) an effective amount of levosimendan, its metabolites OR-1896 or OR-1855, or a combination thereof; (b) a cyclodextrin or a cyclodextrin derivative; and (c) one or more additional pharmaceutically acceptable additives.

In an embodiment, the cyclodextrin derivative comprises an alpha-cyclodextrin derivative, a beta-cyclodextrin derivative, or a gamma-cyclodextrin derivative.

In an embodiment, the cyclodextrin derivative comprises sodium sulfonate salt.

In an embodiment, the cyclodextrin derivative comprises a butyl ether spacer group, an alkyl ether space group, or a combination thereof.

In an embodiment, the cyclodextrin derivative comprises sulfobutylether.

In an embodiment, the cyclodextrin derivative comprises sulfobutylether beta-cyclodextrin.

In an embodiment, the cyclodextrin or cyclodextrin derivative is in an amount of about 50 mg/ml to about 400 mg/ml, preferably in an amount of about 100 mg/ml to about 300 mg/ml.

In an embodiment, the formulation comprises a pH of about 5 to about 9, preferably a pH of about 6 to about 8.

In an embodiment, the one or more pharmaceutically acceptable additives comprise one or more non-citrate buffering agents.

In an embodiment, the one or more pharmaceutically acceptable additives comprise a phosphate buffer.

In an embodiment, the one or more pharmaceutically acceptable additives comprise one or more pH-modifying agents.

In an embodiment, wherein the one or more pharmaceutically acceptable additives comprise one or more preservatives, one or more antioxidants, one or more carriers, or a combination thereof.

In an embodiment, the one or more carriers comprise a liquid media selected from a group consisting of solutions, suspensions, hydrogels, liposomes, emulsions, and a combination thereof.

In an embodiment, the one or more carriers alter the absorption characteristics in a way that extend the effectiveness and or minimize side effects.

In an embodiment, the levosimendan is in an amount of about 0.1 mg/ml to about 100 mg/ml, preferably in an amount of about 0.1 mg/ml to about 30 mg/ml.

In an embodiment, the formulation is substantially free of alcohol.

In an embodiment, the formulation is alcohol-free.

In an embodiment, the formulation is preservative-free.

In an embodiment, the formulation is in the form of particles.

In an embodiment, the formulation is lyophilized.

In an embodiment, the formulation is spray-dried.

In an embodiment, a pharmaceutical composition for subcutaneous administration of levosimendan is in a formulation comprising: (a) levosimendan in an amount of about 0.1 mg/ml to about 10 mg/ml; (b) a cyclodextrin or a cyclodextrin derivative in an amount of about 50 mg/ml to about 500 mg/ml; and (c) phosphate buffer of about 1 mM to about 20 mM.

In an embodiment, the formulation has a pH of about 6 to about 8.

In an embodiment, the formulation is substantially free of alcohol.

In an embodiment, the formulation is lyophilized.

Furthermore, levosimendan formulations that have been described for intravenous administration may be adopted for subcutaneous administration. For example, see U.S. Pat. No. 10,507,179, the entire contents of which are incorporated by reference.

In some embodiments, levosimendan formulations suitable for subcutaneous administration include, but are not limited to, a pharmaceutical composition, comprising levosimendan as active ingredient, and a solubilizer selected from the group consisting of cyclodextrins, consisting of sulfo-butyl-ether beta-cyclodextrin, alpha-cyclodextrin and methyl-beta-cyclodextrin and mixtures thereof, fatty acid esters of glycerol, polyethylene derivatives of alpha-tocopherol, bile acids, with the proviso that the use of co-solvents such as ethanol, propyleneglycol, polyethyleneglycol, poloxamers or polyvinylpyrrolidon is excluded.

In an embodiment, the solubilizer is D-alpha tocopheryl polyethylene glycol 1000 succinate or a bile salt, which is preferably selected from the group consisting of sodium glycocholate, taurocholic acid sodium salt, taurodeoxycholic acid sodium salt and sodium cholate, or mixtures thereof.

In an embodiment, the micelles are polymeric micelles, preferably polyethylene oxide)-pol(propylene oxide) block copolymer micelles, or mixed micelles composed of soy phosphatidylcholine/sodium glycocholate or hybrid micelles.

In an embodiment, the pharmaceutical composition comprises levosimendan as active ingredient, and sulfo-butyl-ether beta-cyclodextrin as a solubilizer, with the proviso that the use of co-solvents comprised of ethanol, propyleneglycol, polyethyleneglycol, poloxamers or polyvinylpyrrolidon is excluded.

In an embodiment, sulfo-butyl-ether beta-cyclodextrin is preferably present in a mmolar ratio compared to levosimendan within a range of 1-15 mmol cyclodextrine(s): 1 mmol levosimendan. Preferably the excess of cyclodextrine(s) is 4-12 mmol cyclodextrine(s); 1 mmol levosimendan, still more preferably 6-10 mmol cyclodextrine(s); 1 mmol levosimendan.

In an embodiment, levosimendan is present in solubilized form.

In an embodiment, levosimendan is solubilized by micellarization or by complexation.

In an embodiment, the pharmaceutical composition is in the form of a solution, more preferably in the form of an aqueous solution.

In an embodiment, the amount of the solubilizer is 2 to 45 percent by weight of the pharmaceutical composition.

In an embodiment, the pH of the solution is in the range of 7.0 to 8.0, more preferably in the range of 7.2 to 7.8.

In an embodiment, the levosimendan in an amount of 1 to 15 mg/ml solution.

In an embodiment, a dried powder is obtained from the pharmaceutical composition for use as a medicament. The dried powder is obtainable by drying a solution comprising the solubilized levosimendan, and is reconstituted to a solution suitable for subcutaneous administration.

In an embodiment, the dried powder for use is a subcutaneous infusion concentrate comprising the levosimendan in an amount of 1 to 15 mg/ml solution.

In an embodiment, the concentrate is to be adjusted to a pH in the range of 7.2 to 8.0.

In an embodiment, the solvent for reconstitution of the dried powder for use is water, or an isotonic buffer system.

In an embodiment, the water has a pH in the range of 7.2 to 7.8, or the isotonic buffer system has a pH in the range of 7.2 to 7.4

In an embodiment, the dried powder is obtainable by drying a solution comprising the solubilized levosimendan and a suitable pharmaceutical vehicle used for freeze-drying.

The present disclosure provides a subcutaneous formulation of levosimendan for use in treating heart failure as part of a combination therapy with SGLT-2, wherein the subcutaneous formulation is obtained from a dried powder, wherein the dried powder is obtained from a pharmaceutical composition comprising: (a) levosimendan; (b) sulfo-butyl-ether beta-cyclodextrin; (c) sodium hydroxide or acetic acid; and water for injection.

In an embodiment, the amount of levosimendan is 2.5 mg/ml water for injection.

In an embodiment, the amount of sulfo-butyl-ether beta-cyclodextrin is 0.175 mg/ml water for injection.

In an embodiment, the sodium hydroxide or acetic acid is in a suitable amount to adjust the pH to a range of 7.2 to 7.8.

In an embodiment, the pharmaceutical composition is filter sterilized.

In an embodiment, the pharmaceutical composition is lyophilized.

In an embodiment, the subcutaneous formulation of levosimendan is obtained from the dried powder by reconstituting the dried powder in an amount of aqueous solution suitable for subcutaneous administration.

In an embodiment, the reconstituted subcutaneous formulation is pH adjusted to 7.2 to 7.8 with sodium hydroxide or acetic acid.

### Oral Administration of Levosimendan

The present invention also relates to oral administration of levosimendan in an oral formulation for use in treating heart failure as part of a combination therapy with SGLT-2. There are numerous practical advantages with oral administration such as: being easily administered and titrated, facilitating patient control, and reducing nursing burden. See, for example, oral formulations of levosimendan provided in PCT International Application Publication No. WO 2021/126884, the entire contents of which are incorporated by reference.

According to some embodiments, oral administration is in the form of hard or soft gelatin capsules, pills, capsules, tablets, including coated tablets, dragees, elixirs, suspensions, liquids, gels, slurries or syrups and controlled release forms thereof. Thus, the invention provides a method of administering levosimendan in the form of a tablet, a capsule, or in a liquid.

Suitable carriers for oral administration are well known in the art. Compositions for oral use can be made using a solid excipient, optionally grinding the resulting mixture, and processing the mixture of granules, after adding suitable auxiliaries as desired, to obtain tablets or dragee cores. Non-limiting examples of suitable excipients include fillers such as sugars, including lactose, sucrose, mannitol, or sorbitol, cellulose preparations such as, maize starch, wheat starch, rice starch, potato starch, gelatin, gum tragacanth, methyl cellulose, hydroxypropylmethyl-cellulose, and sodium carbomethylcellulose, and/or physiologically acceptable polymers such as polyvinylpyrrolidone (PVP).

If desired, disintegrating agents, such as cross-linked polyvinyl pyrrolidone, agar, or alginic acid or a salt thereof, such as sodium alginate, may be added. Capsules and cartridges of, for example, gelatin for use in a dispenser may be formulated containing a powder mix of the compound and a suitable powder base, such as lactose or starch.

Solid dosage forms for oral administration include without limitation capsules, tablets, pills, powders, and granules. In such solid dosage forms, the active compound is admixed with at least one inert pharmaceutically acceptable carrier such as sucrose, lactose, or starch. Such dosage forms can also comprise, as is normal practice, additional substances other than inert diluents, e.g., lubricating, agents. In the case of capsules, tablets and pills, the dosage forms may also comprise buffering agents. Tablets and pills can additionally be prepared with enteric coatings. The term “enteric coating,” as used herein, refers to a coating which controls the location of composition absorption within the digestive system. Non-limiting examples for materials used for enteric coating are fatty acids, waxes, plant fibers or plastics. Liquid dosage forms for oral administration may further contain adjuvants, such as wetting agents, emulsifying and suspending agents, and sweetening, flavoring and perfuming agents.

In an embodiment, the administration is delivered via oral dosing, and the oral dosing can be an immediate release or extended release formulation.

The present disclosure provides a pharmaceutical composition of levosimendan for use in treating heart failure as part of a combination therapy with SGLT-2 by oral administration, wherein the pharmaceutical composition is in a formulation comprising an effective amount of levosimendan, its metabolites OR-1896 or OR-1855, or a combination thereof and one or more additional pharmaceutically acceptable additives.

In an embodiment, the oral formulation comprises levosimendan, its metabolites OR-1896 or OR-1855, or a combination thereof in the amount of 0.1 mg, 0.25 mg, 0.5 mg, 0.75 mg, 1 mg, 2 mg, 3 mg, or 4 mg.

In an embodiment, the oral formulation comprises microcrystalline cellulose.

In an embodiment, the oral formulation comprises alginic acid.

In an embodiment, the oral formulation comprises steric acid.

In an embodiment, the oral formulation is in a capsule form.

In an embodiment, the oral formulation is the capsule form is a HPMC capsule.

In an embodiment the oral formulation comprise in a capsule form and the oral formulation comprises 1 mg levosimendan, 96.4 mg microcrystalline cellulose, 30.0 mg alginic acid, and 5.3 mg stearic acid.

In an embodiment, the oral dosage form comprises levosimendan in the amount of 0.1 mg, 0.25 mg, 0.5 mg, 0.75 mg, 1 mg, 2 mg, 3 mg, or 4 mg, more preferably in the amount of 1-3 mg.

In an embodiment, a subject is orally administered a capsule comprising levosimendan in the amount of 1 mg once per day. The oral dosing may be titrated according to, for example, the effectiveness of the treatment, tolerability, changes in heart rate, and body weight of the subject. The titration of levosimendan may be in 1 mg increments and range from 1-10 mg per day, more preferably between 1-4 mg per day.

The titration of levosimendan administration may occur over the course of days, weeks, or months. The effect of duration at a particular dosage amount on tolerability should also be considered when titrating, e.g. the tolerability of a subject to the levosimendan oral treatment may increase with an increase in duration at a particular dosage amount.

For example, a subject may begin an oral levosimendan treatment course at 1 mg/day (i.e. ingesting one capsule comprising 1 mg levosimendan per day). The subject may maintain a levosimendan dosage of 1 mg/day for two weeks. After two weeks, if the levosimendan dosage is well-tolerated and heart rate is increased <15 BPM, the subject can titrate up to a dosage of 2 mg/day (i.e. ingesting two capsules, each comprising 1 mg levosimendan, per day). The subject may continue to titrate up in increments of 1 mg levosimendan in this manner until an optimal oral dosage is achieved, for example, up to 10 mg of levosimendan per day.

A subject receiving levosimendan by other administration routes, for example, intravenous injection, may be transitioned to an oral dosing scheme. For example, a subject receiving levosimendan by intravenous injection may begin an oral dosing after receiving a final 24-hour infusion of levosimendan. The oral dosing of levosimendan may begin within days or weeks, for example, one week, of the final 24-hour infusion. The oral dosage may begin at 1 mg/day, followed by titration as indicated above.

Each embodiment disclosed herein is contemplated as being applicable to each of the other disclosed embodiments. Thus, all combinations of the various elements described herein are within the scope of the invention.

Examples are provided below to facilitate a more complete understanding of the invention. The following examples illustrate the exemplary modes of making and practicing the invention. However, the scope of the invention is not limited to specific embodiments disclosed in these Examples, which are for purposes of illustration only.

The following examples are presented in order to more fully illustrate some embodiments of the invention. They should, in no way be construed, however, as limiting the broad scope of the invention.

### EXAMPLE

During the HELP Study, as described in PCT International Application Publication No. WO 2021/126884, the entire contents of which are incorporated by reference, Patient 019-002 was administered empagliflozin shortly after beginning levosimendan therapy.

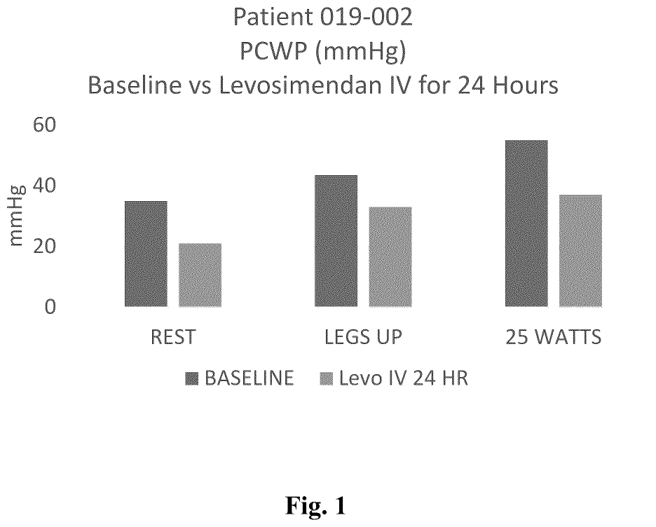

Specifically, Patient 019-002 had established PH-HFpEF and was initially enrolled into the HELP Trial. The diagnosis of PH-HFpEF was confirmed by rest and exercise right heart catheterization. At baseline, the patient had a PCWP at rest of 35 mmHg, and a pulmonary artery pressure of 79/32 mmHg. The PCWP of 35 mmHg at rest increased with exercise to 55 mmHg at baseline. Following 24 hours of i.v. levosimendan, Patient 019-002 had a PCWP at rest of 21 mmHg, which increased with exercise to 37 mmHg. Thus, levosimendan produced a 40% fall in resting PCWP and a 33% fall in exercise PCWP. In addition, at baseline the CVP was 18 mmHg and increased to 30 mmHg with exercise. Following the 24 hours of i.v. levosimendan the CVP fell to 12 mmHg at rest and to 21 mmHg with exercise. Thus, levosimendan produced a 33% fall in resting CVP, and a 30% fall in CVP with exercise. Accordingly, the response of Patient 019-002 to levosimendan treatment was typical of other patients in the study in terms of improvements to their hemodynamic profile (FIG. 1).

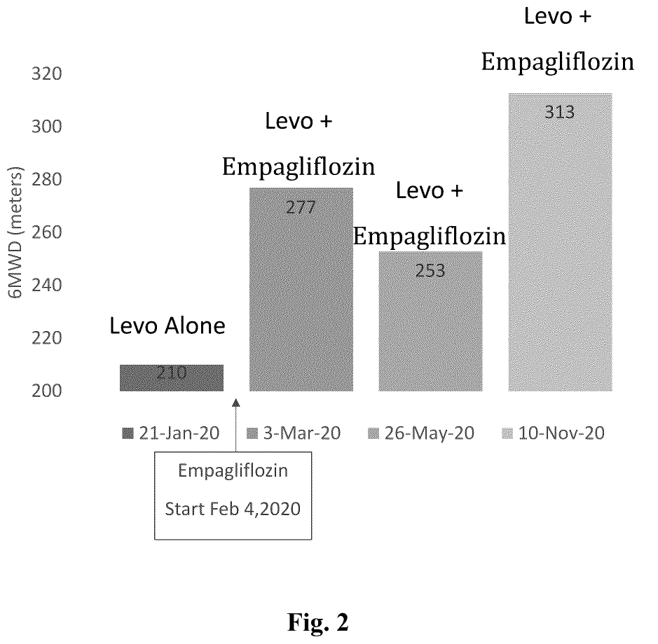

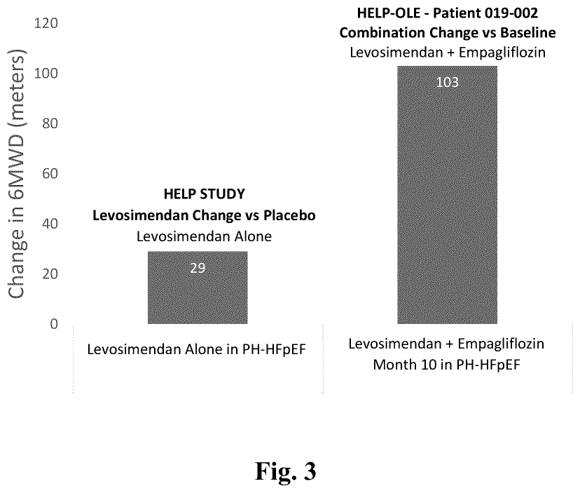

However, the patient showed an unexpectedly dramatic increase in their 6-minute walk distance (6 MWD) upon receiving the combination of levosimendan and empagliflozin (FIG. 2). While the HELP Study found that levosimendan alone improved the 6-minute walk distance of PH-HFpEF patients by approximately 29 m on average, Patient 019-002 increased their 6 MWD by over 100 m when receiving a combination of levosimendan and empagliflozin (FIG. 3). Previous clinical trials that studied treating HFpEF, HFrEF, or PH-HFpEF patients with empagliflozin alone (e.g. EMPERIAL-Preserved, EMPERIAL-Reduced, (Abraham et al.) and EMBRACE HF (Nassif et al.)) showed that the patients did not demonstrate a material change in their exercise capacity as measured by a difference in their 6 MWD. Accordingly, the combination of levosimendan and empagliflozin showed a greater than expected synergistic effect on improving exercise capacity when administered as a heart failure treatment. These results suggest that effective treatment of heart failure, with or without accompanying pulmonary hypertension, can be achieved by a combination therapy of levosimendan and an SGLT-2 inhibitor.

### REFERENCES

Abraham, William T., et al. (2021) “Effect of empagliflozin on exercise ability and symptoms in heart failure patients with reduced and preserved ejection fraction, with and without type 2 diabetes.” European Heart Journal 42.6: 700-710.
Banfor, P. N., et al. (2008). Comparative effects of levosimendan, OR-1896, OR-1855, dobutamine, and milrinone on vascular resistance, indexes of cardiac function, and O2 consumption in dogs. American Journal of Physiology-Heart and Circulatory Physiology, 294(1), H238-H248.
Bittner, B., et al. (2018). Subcutaneous administration of biotherapeutics: an overview of current challenges and opportunities. BioDrugs, 32(5), 425-440.
Borlaug et al. Levosimendan Improves Hemodynamics And Submaximal Exercise Capacity In PH-HFpEF: Primary Results from the Help-PH-HFpEF Multicenter Randomized Controlled Trial Heart Society of America—Late Breaking Clinical Trials Session, Oct. 3, 2020 4:30 PM-5:30 PM.
Burkhoff, Daniel, et al. (2021) “Levosimendan improves hemodynamics and exercise tolerance in PH-HFpEF: results of the randomized placebo-controlled HELP trial.” Heart Failure 9.5: 360-370
Burkhoff et al. “24-hour Levosimendan Infusion Decreases Biventricular Filling Pressures and Increases Cardiac Output at Rest and Exercise in PH-HFpEF.” Circulation 142.Suppl_3 (2020): A15294-A15294.
Chrusciel, P., et al. (2014) Defining the role of trimetazidine in the treatment of cardiovascular disorders: some insights on its role in heart failure and peripheral artery disease. Drugs, 74 (9), 971-980.
De Luca, L., et al. (2006). Effects of levosimendan on left ventricular diastolic function after primary angioplasty for acute anterior myocardial infarction: a Doppler echocardiographic study. Journal of the American Society of Echocardiography, 19(2), 172-177.
Dixon, D. D., et al. (2015). Combined post- and pre-capillary pulmonary hypertension in heart failure with preserved ejection fraction. Heart failure reviews, 21(3), 285-297.
Du Toit, E. F., et al. (2008). A role for the RISK pathway and KATP channels in pre- and post-conditioning induced by levosimendan in the isolated guinea pig heart. British journal of pharmacology, 154(1), 41-50.
ElGuindy, A., et al. (2012). Heart failure with preserved ejection fraction. Global Cardiology Science and Practice, 2012(1), 10.
Erdei, N., et al. (2006). The levosimendan metabolite OR-1896 elicits vasodilation by activating the KATP and BKCa channels in rat isolated arterioles. British journal of pharmacology, 148(5), 696-702.
Galie, N., et al. (2009). Guidelines for the diagnosis and treatment of pulmonary hypertension: the Task Force for the Diagnosis and Treatment of Pulmonary Hypertension of the European Society of Cardiology (ESC) and the European Respiratory Society (ERS), endorsed by the International Society of Heart and Lung Transplantation (ISHLT). European heart journal, 30(20), 2493-2537.
Gorter, T. M., et al. (2018). Right heart dysfunction and failure in heart failure with preserved ejection fraction: mechanisms and management. Position statement on behalf of the Heart Failure Association of the European Society of Cardiology. European journal of heart failure, 20(1), 16-37.
Grossini, E., et al. (2005). Hemodynamic effect of intracoronary administration of levosimendan in the anesthetized pig. Journal of cardiovascular pharmacology. 46(3), 333-342.
Haikala, H., et al. (1995). Mechanisms of action of calcium-sensitizing drugs. Journal of cardiovascular pharmacology, 26, S10-9.
Hajjar, R. J., et al. (1991). Calcium-sensitizing inotropic agents in the treatment of heart failure: a critical view. Cardiovascular drugs and therapy, 5(6), 961-965.
Hasenfuss, G., et al. (1998). Influence of the novel inotropic agent levosimendan on isometric tension and calcium cycling in failing human myocardium. Circulation, 98(20), 2141-2147.
Hawthorne, K. M., et al. (2012). Quality assessment in dobutamine stress echocardiography: what are the clinical predictors associated with a non-diagnostic test?. Cardiology research, 3(2), 73.
Hoeper, M. M., et al. (2016). A global view of pulmonary hypertension. The Lancet Respiratory Medicine, 4(4), 306-322.
Jiang, R., et al. (2018). Efficacy and safety of a calcium sensitizer, levosimendan, in patients with right heart failure due to pulmonary hypertension. The clinical respiratory journal, 12(4), 1518-1525.
Kheinen, P., et al. (2001). Levosimendan increases diastolic coronary flow in isolated guinea-pig heart by opening ATP-sensitive potassium channels. Journal of cardiovascular pharmacology, 37(4), 367-374.
Kelly, J. P., et al. (2015) Patient selection in heart failure with preserved ejection fraction clinical trials. Journal of the American College of Cardiology, 65(16), 1668-1682.
Kivikko, M., et al. (2002). Pharmacodynamics and safety of a new calcium sensitizer, levosimendan, and its metabolites during an extended infusion in patients with severe heart failure. The Journal of Clinical Pharmacology, 42(1), 43-51.
Kivikko, M., et al. (2003). Sustained hemodynamic effects of intravenous levosimendan. Circulation, 107(1), 81-86.
Klapholz, M., et al. (2004). Hospitalization for heart failure in the presence of a normal left ventricular ejection fraction: results of the New York Heart Failure Registry. Journal of the American College of Cardiology, 43(8), 1432-1438.
Kleber, F. X., et al. (2009). Repetitive dosing of intravenous levosimendan improves pulmonary hemodynamics in patients with pulmonary hypertension: results of a pilot study. The Journal of Clinical Pharmacology, 49(1), 109-115.
Kostis, J. B., et al. (2004). Omapatrilat and enalapril in patients with hypertension: the Omapatrilat Cardiovascular Treatment VS. Enalapril (OCTAVE) trial. American journal of hypertension, 17(2), 103-111.
Lai, Y. C., et al. (2019). Insights into the pulmonary vascular complications of heart failure with preserved ejection fraction. The Journal of physiology, 597(4), 1143-1156.
Laurent, S., et al. (2000). Antihypertensive effects of fasidotril, a dual inhibitor of neprilysin and angiotensin-converting enzyme, in rats and humans. Hypertension, 35(5), 1148-1153.
Levine, A. R., et al. (2019). Pulmonary vascular disease in the setting of heart failure with preserved ejection fraction. Trends in cardiovascular medicine, 29(4), 207-217.
Louhelainen, M., et al. (2009). Effects of calcium sensitizer OR-1986 on cardiovascular mortality and myocardial remodelling in hypertensive Dahl/Rapp rats. Acta physiologica Polonica, 12(3), 41.
Louhelainen, M., et al. (2010). Effects of the calcium sensitizer OR-1896, a metabolite of levosimendan, on post-infarct heart failure and cardiac remodelling in diabetic Goto-Kakizaki rats. British journal of pharmacology, 160(1), 142-152.
Maytin, M., et al. (2005). Cardioprotection: a new paradigm in the management of acute heart failure syndromes. The American journal of cardiology, 96(6), 26-31.
Mclaughlin, V. V., et al. (2009). ACCF/AHA 2009 expert consensus document on pulmonary hypertension: a report of the American College of Cardiology Foundation Task Force on expert consensus documents and the American Heart Association developed in collaboration with the American College of Chest Physicians; American Thoracic Society, Inc.; and the Pulmonary Hypertension Association. Journal of the American College of Cardiology, 53(17), 1573-1619.
Michaels, A. D., et al. (2005). Effects of intravenous levosimendan on human coronary vasomotor regulation, left ventricular wall stress, and myocardial oxygen uptake. Circulation, 111(12), 1504-1509.
Nassif, Michael E., et al. (2021) “Empagliflozin Effects on Pulmonary Artery Pressure in Patients With Heart Failure: Results From the EMBRACE-HF Trial.” Circulation 143.17: 1673-1686.
Northridge, D. B., et al. (1999). Comparison of the short-term effects of candoxatril, an orally active neutral endopeptidase inhibitor, and frusemide in the treatment of patients with chronic heart failure. American heart journal, 138(6), 1149-1157.
Norton, G. R., et al. (1999). Sustained Antihypertensive Actions of a Dual Angiotensin-Converting Enzyme Neutral Endopeptidase Inhibitor. Sampatrilat, in Black Hypertensive Subjects. American journal of hypertension, 12(6), 563-571.
Oktay, A. A., et al. (2013). The emerging epidemic of heart failure with preserved ejection fraction. Current heart failure reports, 10(4), 401-410.
Oldroyd, S. H., et al. (2019). Pulmonary Hypertension.
Oudiz, R. J., et al. (2007). Pulmonary hypertension associated with left-sided heart disease. Clinics in chest medicine, 28(1), 233-241.
Opitz, Christian F., et al. (2016). Pre-capillary, combined, and post-capillary pulmonary hypertension: a pathophysiological continuum. Journal of the American College of Cardiology 68.4: 368-378.
Pataricza, J., et al. (2000). Comparison of the vasorelaxing effect of cromakalim and the new inodilator, levosimendan, in human isolated portal vein. Journal of pharmacy and pharmacology, 52(2), 213-217.
Peacock, A. J., et al. (2004). Pulmonary Circulation: Diseases and Their Treatment. CRC Press.
Pollesello, P., et al. (1994). Binding of a new Ca2+ sensitizer, levosimendan, to recombinant human cardiac troponin C. A molecular modelling, fluorescence probe, and proton nuclear magnetic resonance study. Journal of Biological Chemistry, 269(46), 28584-28590.
Pollesello, P., et al. (2007). The cardioprotective effects of levosimendan: preclinical and clinical evidence. Journal of cardiovascular pharmacology, 50(3), 257-263.
Segreti, J. A., et al. (2008). Evoked changes in cardiovascular function in rats by infusion of levosimendan, OR-1896 [(R)-N-(4-(4-methyl-6-oxo-1,4,5,6-tetrahydropyridazin-3-yl)phenyl) acetamide], OR-1855 [(R)-6-(4-aminophenyl)-5-methyl-4,5-dihydropyridazin-3(2H)-one], dobutamine, and milrinone: comparative effects on peripheral resistance, cardiac output, dP/dt, pulse rate, and blood pressure. Journal of Pharmacology and Experimental Therapeutics, 325(1), 331-340.
Shaw, S. J., et al. (2016). Phenotype-specific treatment of heart failure with preserved ejection fraction: a multiorgan roadmap. Circulation, 134(1), 73-90.
Simonneau, G., et al. (2009). Updated clinical classification of pulmonary hypertension. Journal of the American College of Cardiology, 54(1 Supplement), S43-S54.
Sorsa, T., et al. (2004). The contractile apparatus as a target for drugs against heart failure: interaction of levosimendan, a calcium sensitiser, with cardiac troponin c. Molecular and cellular biochemistry, 266(1-2), 87-107.
Szilagyi, S., et al. (2004). The effects of levosimendan and OR-1896 on isolated hearts, myocyte-sized preparations and phosphodiesterase enzymes of the guinea pig. European journal of pharmacology, 486(1), 67-74.
Teerlink, J. R., et al. (2016). RELAX-REPEAT: a multicenter, prospective, randomized, double-blind study evaluating the safety and tolerability of repeat doses of serelaxin in patients with chronic heart failure. Journal of Cardiac Failure, 22(8), S14-S15.
Torres, F., et al. (2019). Efficacy and safety of ralinepag, a novel oral IP agonist, in PAH patients on mono or dual background therapy: results from a phase 2 randomised, parallel group, placebo-controlled trial. European Respiratory Journal, 54(4).
Vandoni, M., et al. (2018). Six minute walk distance and reference values in healthy Italian children: A cross-sectional study. PloS one, 13(10).
Virgadamo, S., et al. (2015). Digoxin: A systematic review in atrial fibrillation, congestive heart failure and post myocardial infarction. World journal of cardiology, 7(11), 808.
Wait, J. C., et al. (2006). Metabolism of [14C] gemopatrilat after oral administration to rats, dogs, and humans. Drug metabolism and disposition, 34(6), 961-970.
Yancy, C. W., et al. (2013). 2013 ACCF/AHA guideline for the management of heart failure: a report of the American College of Cardiology Foundation/American Heart Association Task Force on Practice Guidelines. Journal of the American College of Cardiology, 62(16), e147-e239.
Yancy, C. W., et al. (2013). 2013 ACCF/AHA guideline for the management of heart failure: executive summary: a report of the American College of Cardiology Foundation/American Heart Association Task Force on Practice Guidelines. Circulation, 128(16), 1810-1852.
Yokoshiki, H., et al. (1997). Levosimendan, a novel Ca2+ sensitizer, activates the glibenclamide-sensitive K+ channel in rat arterial myocytes. European journal of pharmacology, 333(2-3), 249-259.

1. A method of treating heart failure in a human subject comprising administering to the human subject an effective amount of a combination therapy comprising
a) an amount of levosimendan, its metabolites OR-1896 or OR-1855, or a combination thereof; and
b) an amount of a sodium-glucose cotransporter-2 (SGLT-2) inhibitor.

2. The method of claim 1, comprising periodically administering to the subject an amount of the levosimendan, its metabolites OR-1896 or OR-1855, or a combination thereof, and an amount of the SGLT-2 inhibitor, wherein the amounts when taken together are effective to treat the subject;
wherein treating the subject with the combination therapy is more effective to treat the subject than when either the amount of levosimendan or the amount of the SGLT-2 inhibitor is administered alone; and/or
wherein the amounts of levosimendan and the SGLT-2 inhibitor when taken together are effective to achieve a greater than additive therapeutic result in treating the subject.

3-4. (canceled)

5. The method of claim 1, wherein the subject was receiving a therapy including levosimendan, its metabolites OR-1896 or OR-1855, or a combination thereof, prior to initiating a SGLT-2 inhibitor therapy; or
wherein the subject was receiving a SGLT-2 inhibitor therapy prior to initiating a therapy including levosimendan, its metabolites OR-1896 or OR-1855, or a combination thereof.

6. (canceled)

7. The method claim 1, wherein the amount of SGLT-2 inhibitor is administered first, followed by administration of the amount of levosimendan, its metabolites OR-1896 or OR-1855, or a combination thereof; or
wherein the amount of levosimendan, its metabolites OR-1896 or OR-1855, or a combination thereof, is administered first, followed by administration of a SGLT-2 inhibitor.

8. (canceled)

9. The method of claim 1, wherein the levosimendan, its metabolites OR-1896 or OR-1855, or a combination thereof, and the SGLT-2 inhibitor are administered sequentially; or
wherein the levosimendan, its metabolites OR-1896 or OR-1855, or a combination thereof, and the SLGT-2 inhibitor are administered simultaneously.

10. (canceled)

11. The method of claim 1, wherein the levosimendan, its metabolites OR-1896 or OR-1855, or a combination thereof, and the SLGT-2 inhibitor are administered periodically, chronically, weekly, or intermittently.

12. The method of claim 11, wherein the SGLT-2 inhibitor is administered orally; and/or
the group wherein the SGLT-2 inhibitor is selected from consisting of empagliflozin, canagliflozin, ertugliflozin, or dapagliflozin;
preferably wherein the subject is administered between 10-25 mg empagliflozin per day;
preferably wherein the subject is administered between 5-10 mg dapagliflozin per day;
preferably wherein the subject is administered between 100-300 mg canagliflozin per day; or
preferably wherein the subject is administered between 5-15 mg ertugliflozin per day.

13-17. (canceled)

18. The method of claim 1, wherein the levosimendan, its metabolites OR-1896 or OR-1855, or a combination thereof, is administered orally, intravenously, or subcutaneously.

19. The method of claim 1, wherein the subject is administered between 0.1-10 mg of levosimendan, its metabolites OR-1896 or OR-1855, or a combination thereof, per day, preferably between 1-4 mg of levosimendan, its metabolites OR-1896 or OR-1855, or a combination thereof per day.

20. The method of claim 1, wherein the combination therapy is administered as a fixed dose combination.

21. The method of claim 1, wherein the heart failure is heart failure with preserved ejection fraction (HFpEF) or heart failure with reduced ejection fraction (HFrEF).

22. The method of claim 21, wherein the heart failure is accompanied by pulmonary hypertension.

23. The method of claim 22, wherein the heart failure accompanied by pulmonary hypertension is pulmonary hypertension with heart failure with preserved ejection fraction (PH-HFpEF) or pulmonary hypertension with heart failure with reduced ejection fraction (PH-HFrEF).

24. The method of claim 1, wherein the treating with the combination therapy comprises providing
a) an improvement in the human subject's quality of life;
b) an improvement in the human subject's exercise capacity;
c) an improvement in a physician's assessment of the human subject's functional class; and/or
d) a reduction in the incidence of hospitalization for heart failure.

25. The method of claim 24, wherein the treating with the combination therapy comprises providing an improvement in the human subject's exercise capacity.

26. The method of claim 25, wherein the improvement in the subject's exercise capacity is an increase of at least 10, 20, 30, 40, 50, 60, 70, 80, or 100 meters in a 6-minute walk distance compared to a baseline 6-minute walk distance before the combination therapy treatment; and/or
wherein the improvement in the subject's exercise capacity is an increase of at least 10%, 20%, 30%, 40%, or 50% relative to a baseline 6-minute walk distance before the combination therapy treatment.

27. (canceled)

28. The method of claim 25, wherein the improvement in the subject's exercise capacity is within one, two, three, four, five, six, seven, eight, nine, ten, twenty, thirty, forty, or fifty weeks of the administration of the combination therapy.

29. The method of claim 1, wherein the treating comprises providing an improvement in the human subject's hemodynamic measurements at rest and exercise.

30. A pharmaceutical composition comprising levosimendan, its metabolites OR-1896 or OR-1855, or a combination thereof, a SGLT-2 inhibitor, and a pharmaceutically acceptable carrier, preferably wherein the SGLT-2 inhibitor is empagliflozin; or
a pharmaceutical composition comprising an amount of a SGLT-2 inhibitor and an amount of levosimendan, its metabolites OR-1896 or OR-1855, or a combination thereof, for use in treating a subject afflicted with heart failure, wherein the SGLT-2 inhibitor and the levosimendan, its metabolites OR-1896 or OR-1855, or a combination thereof, are administered simultaneously, contemporaneously or concomitantly;
preferably wherein the heart failure is heart failure with preserved ejection fraction (HFpEF), heart failure with reduced ejection fraction (HFrEF), pulmonary hypertension with heart failure with preserved ejection fraction (PH-HFpEF) or pulmonary hypertension with heart failure with reduced ejection fraction (PH-HFrEF); and/or
preferably wherein the SGLT-2 inhibitor is selected from the group consisting of empagliflozin, canagliflozin, ertugliflozin, or dapagliflozin; or
a pharmaceutical composition in unit dosage form, useful in treating a subject afflicted with heart failure, which comprises:
e) an amount of levosimendan; and
f) an amount of an SGLT-2 inhibitor,
wherein the respective amounts of said levosimendan and said SGLT-2 inhibitor in said composition are effective, upon concomitant administration to said subject of one or more said unit dosage forms of said composition, to achieve a greater than additive therapeutic result in treating the subject;
preferably wherein the SGLT-2 inhibitor is selected from the group consisting of empagliflozin, canagliflozin, ertugliflozin, or dapagliflozin, and the heart failure is heart failure with preserved ejection fraction (HFpEF), heart failure with reduced ejection fraction (HFrEF), pulmonary hypertension with heart failure with preserved ejection fraction (PH-HFpEF) or pulmonary hypertension with heart failure with reduced ejection fraction (PH-HFrEF); or
a package comprising:
a) a first pharmaceutical composition comprising an amount of levosimendan and a pharmaceutically acceptable carrier;
b) a second pharmaceutical composition comprising an amount of an SGLT-2 inhibitor and a pharmaceutically acceptable carrier; and
c) instructions for use of the first and second pharmaceutical compositions together to treat a subject afflicted with heart failure;
preferably wherein the SGLT-2 inhibitor is empagliflozin and the heart failure is heart failure with preserved ejection fraction (HFpEF), heart failure with reduced ejection fraction (HFrEF), pulmonary hypertension with heart failure with preserved ejection fraction (PH-HFpEF) or pulmonary hypertension with heart failure with reduced ejection fraction (PH-HFrEF); or
a therapeutic package for dispensing to, or for use in dispensing to, a subject afflicted with heart failure, which comprises:
a) one or more unit doses, each such unit dose consisting essentially of:
i) an amount of levosimendan; and
ii) an amount of an SGLT-2 inhibitor,
wherein the respective amounts of said levosimendan and said SGLT-2 inhibitor in said unit dose are effective, upon concomitant administration to said subject, to achieve a greater than additive therapeutic result in treating the subject, and
b) a finished pharmaceutical container therefor, said container containing said unit dose or unit doses, said container further containing or comprising labeling directing the use of said package in the treatment of said subject;
preferably wherein the SGLT-2 inhibitor is selected from the group consisting of empagliflozin, canagliflozin, ertugliflozin, or dapagliflozin, and the heart failure is heart failure with preserved ejection fraction (HFpEF), heart failure with reduced ejection fraction (HFrEF), pulmonary hypertension with heart failure with preserved ejection fraction (PH-HFpEF) or pulmonary hypertension with heart failure with reduced ejection fraction (PH-HFrEF).

31. (canceled)

32. Use of a SGLT-2 inhibitor in combination or as an add-on with a therapy that includes levosimendan, its metabolites OR-1896 or OR-1855, or a combination thereof, to treat a subject afflicted with heart failure, wherein the SGLT-2 inhibitor and the levosimendan, its metabolites OR-1896 or OR-1855, or a combination thereof are administered simultaneously, contemporaneously or concomitantly;
preferably wherein the SGLT-2 inhibitor is selected from the group consisting of empagliflozin, canagliflozin, ertugliflozin, or dapagliflozin; and/or
preferably wherein the heart failure is heart failure with preserved ejection fraction (HFpEF), heart failure with reduced ejection fraction (HFrEF), pulmonary hypertension with heart failure with preserved ejection fraction (PH-HFpEF) or pulmonary hypertension with heart failure with reduced ejection fraction (PH-HFrEF); or
use of a SGLT-2 inhibitor in the manufacturing of a medicament for use in combination with or as an add-on to a therapy that includes levosimendan, its metabolites OR-1896 or OR-1855, or a combination thereof, to treat a subject afflicted with heart failure, wherein the SGLT-2 inhibitor and the levosimendan, its metabolites OR-1896 or OR-1855, or a combination thereof are administered simultaneously, contemporaneously or concomitantly;
preferably wherein the heart failure is heart failure with preserved ejection fraction (HFpEF), heart failure with reduced ejection fraction (HFrEF), pulmonary hypertension with heart failure with preserved ejection fraction (PH-HFpEF) or pulmonary hypertension with heart failure with reduced ejection fraction (PH-HFrEF); and/or
preferably wherein the SGLT-2 inhibitor is selected from the group consisting of empagliflozin, canagliflozin, ertugliflozin, or dapagliflozin.

33-46. (canceled)
